(19) United States
(12) Patent Application Publication
(10) Pub. No.: US 2024/0101659 A1
(43) Pub. Date: Mar. 28, 2024

(54) STABLE THERAPEUTIC PROTEIN FORMULATION AND METHODS OF MAKING THE SAME

(71) Applicant: DR. REDDY’S LABORATORIES LIMITED, Hyderabad (IN)

(72) Inventors: Murali JAYARAMAN, Kancheepuram (Dist) (IN); Swapnil Vasudeo PAKHALE, Jalgaon Dist (IN); Vikram KAITHWAS, Ujjain (IN)

(21) Appl. No.: 18/038,282

(22) PCT Filed: Nov. 24, 2021
(86) PCT No.: PCT/IN2021/051095

§ 371 Date: May 23, 2023

(30) Foreign Application Priority Data
Nov. 25, 2020 (IN) 202041051228

(51) Int. Cl. C07K 16/24; A61K 47/12; A61K 47/18; A61K 47/26

(52) U.S. Cl. CPC C07K 16/244; A61K 47/12; A61K 47/183; A61K 47/26

(57) ABSTRACT

The present invention discloses a pharmaceutical formulation of antibody that binds to interleukin. The invention also discloses methods of making the same. The formulation composition comprises a weak organic acid and organic base buffer and pharmaceutically acceptable excipients. The said composition stabilizes the antibody by controlling aggregation, degradation, oxidation and formation of charge variants. The disclosed antibody formulations are liquid formulations that are also suitable for lyophilization.

### FIELD OF THE INVENTION

The present invention is related to a stable pharmaceutical formulation of an anti-interleukin-17 antibody molecule, wherein the antibody is stabilized by a specific combination of buffer and excipients. The disclosed formulations are compatible with lyophilized as well as liquid form.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, although considered more stable than liquid (aqueous) formulations, but reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc. All of these stress factors could result in the loss of biological activity of a therapeutic protein/antibody.

In particular, high concentration liquid formulations are susceptible to degradation and/or aggregation. Nevertheless, high concentration formulations may be desirable for subcutaneous route of administration, as the frequency of administration and injection volume is reduced.

Among many US Food and Drug Administration (FDA) approved high concentration antibodies, anti-interleukin-17 antibodies (secukinumab, ixekizumab and brodalumab) are one, wherein the concentration of these antibodies ranges from 80 mg/ml to 150 mg/ml. However, and as stated earlier, as the concentration of protein goes higher in a formulation, the tendency of protein aggregation and degradation is more which leads to the instability of the protein. It has been reported that, anti-interleukin-17 antibodies (secukinumab) are prone for oxidation (specially, at cys97 in light chain of the antibody, line numbers 1-10, page number 2 of WO2016103146).

Hence, the objective of the present invention is to develop a stable anti-interleukin-17 antibody, wherein the antibody formulated in a specific formulation composition stabilizes the antibody by reducing/inhibiting the formation of aggregates, fragments, charge variants and oxidized species of the antibody.

### SUMMARY

The present invention discloses an aqueous pharmaceutical formulation of an anti-interleukin-17 antibody comprising, anti-interleukin-17 antibody, and a buffer composition comprising a combination of weak organic acid and organic base. The anti-interleukin-17 antibody formulated in the above said buffer is stable and, maintains greater than 90% of monomeric content of the antibody even after storage for two weeks at 40° C.

Further, the anti-interleukin-17 antibody formulations disclosed in the invention contain sugar(s), amino acid(s) and/or surfactant.

In particular, the invention discloses a high concentration aqueous pharmaceutical formulation of an anti-interleukin-17 antibody, comprising 150 mg/ml anti-interleukin-17 antibody, a buffer comprising a combination of weak organic acid and organic base, sugar, amino acid and surfactant. The combination of sugar and amino acid in the said buffer aids in the inhibition of formation of aggregates and/or charge variants of the antibody present in the said formulation.

In one aspect, the invention discloses a method of controlling fragmentation and/or formation of charge variants of an anti-interleukin-17 antibody in the formulation composition of the antibody, the method comprising formulating the antibody in a composition comprising a buffer which is a combination of weak organic acid and organic base, sugar, and amino acid(s).

In another aspect, invention discloses a method of controlling oxidation of an anti-interleukin-17 antibody in the formulation composition of the antibody, the method comprising formulating the antibody in a composition comprising a weak organic acid and organic base buffer, sugar, and arginine, wherein the formulation does not require any anti-oxidant or methionine.

The antibody in the said formulation is stable and maintains greater than 92% of monomeric content of the antibody in the formulation even after storage for two weeks at 40° C.

In yet another aspect, the invention discloses a method of controlling aggregation and/or deamidation of an anti-interleukin-17 antibody in the formulation composition of the antibody, the method comprising formulating the antibody in a composition comprising a weak organic acid and organic base buffer, sugar, arginine and surfactant.

The disclosed anti-interleukin-17 antibody formulations do not require an anti-oxidant or methionine to stabilize the antibody.

The disclosed liquid formulations are stable at room temperature/accelerated storage conditions (at 40° C.) and compatible with lyophilization process.

### DETAILED DESCRIPTION OF THE INVENTION

### Definitions

The term “about” refers to a range of values that are similar to the stated reference value and includes a range of values that fall within 20% or less, of the stated reference value.

The term “antibody” refers to a glycoprotein comprising at least two heavy (H) chains and two light (L) chains inter-connected by disulfide bonds, or an antigen-binding portion thereof. The “antibody” as used herein encompasses whole antibodies or any antigen binding fragment (i.e., “antigen-binding portion”) or fusion protein thereof.

The term “pre-formulation” refers to any or multiple steps performed before formulating the protein into a therapeutic product. Examples of such steps include, chromatography, filtration, (ultrafiltration, sterile filtration, nano filtration, diafiltration, depth filtration), or any other steps performed to concentrate the protein or to exchange the buffer to a different/suitable buffer. The filtration steps mentioned herein may be performed in a tangential flow filtration mode.

The term “stable” formulation refers to the formulation wherein the antibody therein retains its physical stability and/or chemical stability and/or biological activity.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Positively charged molecules bind to anion exchange resins while negatively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof.

The term sugar/s as used herein includes sugars and sugar alcohols or polyols. Sugars can be referred to monosaccharides, disaccharides, and polysaccharides. Examples of sugars include, but are not limited to, sucrose, trehalose, glucose, dextrose, raffinose and others. Examples of sugar alcohols or polyols include, but are not limited to, mannitol, sorbitol, and others.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

The term “amino acid” as used herein refers to amino acid that is included in the formulation and is not a part of the buffer component. An amino acid may be present in its D- and/or L- form.

The term “antioxidant” refers to an agent that inhibits the oxidation of other molecules and is not part of buffer component. Examples of antioxidants herein include methionine, citrate, lipoic acid, uric acid, glutathione, tocopherol, carotene, lycopene, cysteine, phosphonate compounds, e.g., etidronic acid, desferoxamine and malate.

The term “charge variants” herein refers to an antibody variants which has net positive or negative charge and contains either lower or higher isoelectric point (pI) than the antibody of interest. Examples of charge variants include acidic variants and basic variants. The acidic variants of an antibody can be formed due to deamidation of glutamine and aspargine and sialylation which may impart net negative charge to the antibody and resulted in decrease in pI of the antibody. The basic variants of an antibody can be formed due to C-terminal lysine variation, oxidation, glycine amidation, succinamide formation, removal of sialic acids which may impart net positive charge to the antibody and resulted in increase in pI of the antibody.

### Detailed Description of Embodiments

The instant invention discloses an antibody formulation, specifically anti-interleukin-17 antibody formulation, which is stabilized by a buffer composition comprising a combination of weak organic acid and organic base.

The present invention discloses a high concentration aqueous pharmaceutical formulation of an anti-interleukin-17 antibody comprising an anti-interleukin-17 antibody and a buffer comprising a combination of weak organic acid and organic base having pH of about 5.5 to about 6.5.

The instant invention discloses an antibody formulation, specifically anti-interleukin-17 antibody formulation, which is stabilized by a composition comprising an organic base buffer, arginine, sugar and surfactant. The said composition does not require methionine or an anti-oxidant to stabilize the anti-interleukin-17 antibody formulation.

In the above embodiment, the concentration of the antibody is from about 150 mg/ml to about 170 mg/ml.

In any of the above embodiment, the concentration of the antibody is about 150 mg/ml.

In one embodiment, the invention discloses a method of inhibiting aggregation of an anti-interleukin-17 antibody in the formulation composition of the antibody, the method comprising, formulating the antibody in a buffer composition comprising a combination of weak organic acid and organic base, having pH of 5.5 to 6.5.

In another embodiment, the invention discloses a method of inhibiting fragmentation of an anti-interleukin-17 antibody in the formulation composition of the antibody, the method comprising formulating the antibody in a buffer composition comprising a combination of weak organic acid and organic base, having pH of 5.5 to 6.5.

In another embodiment, the invention discloses a method of inhibiting formation of charge variants of an anti-interleukin-17 antibody in the formulation composition of the antibody, the method comprising, formulating the antibody in a buffer composition comprising a combination of weak organic acid and organic base, having pH of 5.5 to 6.5.

In yet another embodiment, the invention discloses a method of controlling oxidation of an anti-interleukin-17 antibody in the formulation composition of the antibody, the method comprising formulating the antibody in a buffer composition comprising a combination of weak organic acid and organic base, having pH of 5.5 to 6.5.

In an embodiment, the invention discloses a method of imparting colloidal stability to an anti-interleukin-17 antibody in the formulation composition of the antibody, the method comprising formulating the antibody in a buffer composition comprising a combination of weak organic acid and organic base, having pH of 5.5 to 6.5.

In any of the above embodiments, the organic base is histidine.

In any of the above embodiments, the weak organic acid is acetate or citrate.

In any of the above mentioned embodiments, the formulations may further comprise pharmaceutical acceptable excipients such as sugars, amino acid/s, salt, and surfactant.

In one embodiment, the invention discloses an aqueous high concentration anti-interleukin-17 antibody formulation comprising;
a) 150 mg/ml of anti-interleukin-17 antibody
b) a buffer composition comprising a combination of weak organic acid and organic base having a pH value ranging from 5.5 to 6.5,
c) one or more of the following excipients
   i) sugar,
   ii) amino acid
   iii) surfactant

In the above mentioned embodiment, the anti-interleukin-17 antibody formulation is devoid of anti-oxidant.

In an embodiment, the invention discloses a method of inhibiting aggregation of an anti-interleukin-17 antibody in the formulation composition of the antibody, the method comprising, formulating the antibody in a buffer composition comprising weak organic acid and organic base, sugar, amino acid/s and surfactant.

In an embodiment, the invention discloses a method of controlling aggregation of an anti-interleukin-17 antibody in the formulation composition of the antibody, the method comprising formulating the antibody in an organic base buffer composition comprising, sugar and arginine, wherein the pH of the formulation is about 6, and the antibody formulated by the said method contains less than 5% aggregate content when the sample is stored at 40° C. for two weeks.

In an embodiment, the invention discloses a method of controlling formation of charge-variants of an anti-interleukin-17 antibody in the formulation composition of the antibody, the method comprising formulating the antibody in a buffer composition comprising weak organic acid and organic base, sugar, amino acid/s and surfactant.

In another embodiment, the invention discloses a method of controlling oxidation of an anti-interleukin 17 antibody in the formulation composition of the antibody, the method comprising formulating the antibody in a buffer composition comprising weak organic acid and organic base, sugar, arginine and surfactant, wherein the formulation does not require any other anti-oxidant or methionine to stabilize the antibody.

In yet another embodiment, the invention discloses a method of inhibiting fragmentation of an anti-interleukin-17 antibody in the formulation composition of the antibody, the method comprising formulating the antibody in a buffer composition comprising weak organic acid and organic base, sugar, amino acid and surfactant.

In an embodiment, the invention discloses a method of controlling fragmentation of an anti-interleukin-17 antibody in the formulation composition of the antibody, the method comprising formulating the antibody in an organic base buffer composition comprising, sugar and arginine, wherein the pH of the formulation is about 6 and the antibody formulated by the said method contains less than 3% as fragments of the antibody when the sample is stored for two weeks at 40° C.

In one embodiment, the invention discloses a method of stabilizing an anti-interleukin-17 antibody, comprising addition of a buffer composition comprising weak organic acid and organic base, sugar and amino acid/s during pre-formulation stages of antibody processing, followed by formulating the antibody in a buffer comprising weak organic acid and organic base, sugar, amino acid and surfactant, wherein the antibody formulated in the said composition is stable as measured by its aggregates/variants or fragments content.

In another embodiment, the invention discloses an aqueous anti-interleukin-17 antibody formulation suitable for subcutaneous administration to a patient, the formulation comprising about 150 mg/ml anti-interleukin-17 antibody, a weak organic acid and organic base buffer, sugar, arginine and surfactant, wherein the pH of the formulation is about 6 and wherein the formulation does not contain an anti-oxidant/methionine.

In another embodiment, the invention discloses an anti-interleukin-17 antibody formulation suitable for subcutaneous administration to a patient, the formulation comprising; about 150 mg/ml of anti-interleukin-17 antibody; histidine buffer, sugar, arginine and surfactant, wherein the pH of the formulation is about 6, and wherein the formulation does not require any antioxidant or methionine to stabilize the antibody.

In another embodiment, the invention discloses an anti-interleukin-17 antibody formulation suitable for subcutaneous administration to a patient, the formulation comprising; about 150 mg/ml of anti-interleukin-17 antibody; histidine-acetate buffer, sugar, arginine and surfactant, wherein the pH of the formulation is about 6, and wherein the formulation does not require any antioxidant/methionine to stabilize the antibody.

In yet another embodiment, the invention discloses an anti-interleukin-17 antibody formulation suitable for subcutaneous administration to a patient, the formulation comprising; about 150 mg/ml of anti-interleukin-17 antibody; histidine-citrate buffer, sugar, arginine and surfactant, wherein the pH of the formulation is about 6, and the formulation does not require any antioxidant/methionine to stabilize the antibody.

In any of the above mentioned embodiments, the anti-interleukin-17 antibody formulation may optionally contain a chelating agent.

In the above mentioned embodiment, the chelating agent is ethylene diamine tetra acetic acid (EDTA), diethylenetriaminepentaacetic acid (DTPA), nitrilotriacetic acid (NTA), N-2-acetamido-2—iminodiacetic acid (ADA), bis(aminoethyl)glycol ether, N,N,N′,N′-tetraacetic acid (EGTA), trans- diaminocyclohexane tetraacetic acid (DCTA), N- hydroxyethyliminodiacetic acid (HIMDA), N,N-bis-hydroxyethylglycine (bicine), N- (trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid and malate.

In any of the above mentioned embodiments, the combination of sugar and amino acid/s in the said buffer protects the anti-interleukin-17 antibody from various physical and chemical instability.

In any of the above mentioned embodiments, the formulation is stable and maintains greater than 90% of the antibody in monomeric form even after storage at 40° C. for two weeks.

In any of the above mentioned embodiments, the interleukin-17 antibody formulations are stable for two weeks at 40° C. and are free of any visible particles.

In an embodiment, the invention discloses an aqueous anti-interleukin-17 antibody formulation comprising;
a) about 150 mg/ml anti-interleukin-17 antibody
b) a buffer composition comprising a combination of weak organic acid and organic base, having pH of about 6,
c) about 200 mM sugar
d) about 10 mM arginine
e) 0.02% (w/v) surfactant.

In the above said embodiment, the formulation is substantially free from visible particles upon storage at about 40° C. for at least two weeks as determined by visual inspection.

In the above said embodiment, the combination of sugar, buffer and amino acid present in the formulation aids in controlling formation of acidic variant content, aggregation and oxidation of the antibody molecule, during storage.

In any of the above mentioned embodiments, the surfactant is polysorbate 20 or polysorbate 80.

In any of the above mentioned embodiments, the disclosed anti-interleukin-17 antibody formulations of the present invention does not require any antioxidant or methionine.

In any of the above mentioned embodiments, the pH of the anti-interleukin-17 antibody formulations remains stable even after storage under accelerated conditions such as at 40° C. for two weeks.

In any of the above mentioned embodiments, the viscosity of the anti-interleukin-17 antibody formulations is less than 20 cP.

In any of the above mentioned embodiments, the high concentration formulation of an anti-interleukin-17 antibody include about 75 mg/ml or about 100 mg/ml or about 125 mg/ml or about 150 mg/ml or about 160 mg/ml or about 170 mg/ml of anti-interleukin 17 antibody.

In any of the above mentioned embodiments, the anti-interleukin-17 antibody is secukinumab.

In any of the above mentioned embodiments, the formulation of an anti-interleukin-17 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of an anti-interleukin-17 antibody can be reconstituted with appropriate diluent to achieve the liquid formulation suitable for administration.

In any of the above mentioned embodiments, the stable liquid interleukin-17 antibody formulations are compatible with lyophilization process and the lyophilization process does not impact the quality attributes of the antibody.

Another aspect of the invention provides a vial, pre-filled syringe or an auto injector device, or any other suitable device comprising any of the subject formulations described herein. In certain embodiments, the aqueous formulation stored in the vial or pre-filled syringe or auto injector device contains an anti-interleukin-17 antibody, buffer, sugar, amino acid and surfactant.

Certain specific aspects and embodiments of the invention are more fully described by reference to the following examples. However, these examples should not be construed as limiting the scope of the invention in any manner.

### Examples

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

An anti-interleukin-17 antibody, secukinumab, suitable for storage in the present pharmaceutical composition is produced by standard methods known in the art. For example, secukinumab is prepared by recombinant expression of immunoglobulin light and heavy chain genes in a mammalian host cell such as Chinese Hamster Ovary cells. Further, the expressed secukinumab is harvested and the crude harvest is subjected to standard downstream process steps that include purification, filtration and optionally dilution or concentration steps. For example, the crude harvest of secukinumab may be purified using standard chromatography techniques such as affinity chromatography, ion-exchange chromatography and combinations thereof. The purified secukinumab solution can additionally be subjected to one or more filtration steps, and the solution obtained is subjected to further formulation studies.

### Example 1: Secukinumab Formulations in Various Buffer Back Ground

Secukinumab (at concentration 30.02 mg/ml) obtained from final step of downstream process, was buffer exchanged into various buffers (listed in Table-1) and concentrated up to 160 mg/ml-170 mg/ml in the said buffers.

All secukinumab formulations were subjected for accelerated stability studies at 40° C. for two weeks. Post which, the samples were analyzed for high molecular weight (UMW) species and monomer content using size exclusion chromatography (SEC) [results are given in Table 2] and also, these samples were checked for change in pH. It was observed that, there was no change in the pH after storage at 40° C. for two weeks.

TABLE 1
Buffer compositions used in example 1 to prepare
high concentration secukinumab formulations.

| Sample Name | Composition |
|---|---|
| Smab-C | 20 mM histidine buffer, pH 5.8 |
| Smab-1 | 20 mM citrate-phosphate buffer, pH 5.8 |
| Smab-2 | 20 mM citrate buffer, pH 5.8 |
| Smab-3 | 20 mM histidine-citrate buffer, pH 5.8 |
| Smab-4 | 20 mM histidine-acetate buffer, pH 5.8 |

TABLE 2
SEC data of high concentration secukinumab (~160
mg mg/ml) formulations prepared as per example 1

| Sam- | SEC data at 40° C. | | | | | | | | |
|---|---|---|---|---|---|---|---|---|---|
| ple | % of monomer | | | % of LMW | | | % of HMW | | |
| Name | 0 W | 1 W | 2 W | 0 W | 1 W | 2 W | 0 W | 1 W | 2 W |
| Smab-C | 99.1 | 96.1 | 94.8 | 0.0 | 0.3 | 0.5 | 0.9 | 3.6 | 4.7 |
| Smab-1 | 98.8 | 95.7 | 94.4 | 0.0 | 0.2 | 0.4 | 1.2 | 4.1 | 5.2 |
| Smab-2 | 98.9 | 95.9 | 94.6 | 0.0 | 0.2 | 0.4 | 1.1 | 3.9 | 5.1 |
| Smab-3 | 99.1 | 96.3 | 95.1 | 0.0 | 0.2 | 0.4 | 0.9 | 3.5 | 4.5 |
| Smab-4 | 99.0 | 96.0 | 94.6 | 0.0 | 0.3 | 0.5 | 1.0 | 3.7 | 4.9 |

W—indicates week

### Example 2: Secukinumab Formulations in Combination with Different Excipients without an Anti-Oxidant

Purified secukinumab obtained from downstream chromatographic steps was buffer exchanged into various buffers and then concentrated up to 150 mg/ml. Post which, sugar/polyol such as trehalose/mannitol, amino acid (arginine), and polysorbate were added to prepare final formulations (Table 3). And also, cysteine/EDTA were added to few of the samples to know the effect of these excipients on the stability of secukinumab formulations. To maintain a positive control, 150 mg/ml secukinumab was formulated in the approved formulation composition comprising histidine buffer containing 200 mM trehalose, 10 mM methionine, 0.02% (w/v). The approved formulation contains methionine (an anti-oxidant) for the stability of secukinumab.

All secukinumab formulations were subjected for accelerated stability studies at 40° C. for two weeks. Post which, the samples were analyzed for high molecular weight (UMW) species and monomer content using size exclusion chromatography (SEC) [results are given in Table 4] and main peak content/acidic variants using Ion exchange chromatography [results are given in Table 5]. Further, these sample were monitored for changes in pH, colour/appearance and for any visible particles.

TABLE 3
High concentration secukinumab formulations prepared as per example 2

| Sample Name | Composition |
|---|---|
| Smab-Control | 150 mg/ml secukinumab, 20 mM histidine, 200 mM trehalose |
| | dihydrate, 10 mM methionine, 0.02% polysorbate 80, pH 5.8 |
| Smab-5 | 150 mg/ml secukinumab, 20 mM histidine, 200 mM trehalose |
| | dihydrate, 10 mM di-sodium EDTA, 10 mM arginine 0.02% |
| | polysorbate 80, pH 5.8 |
| Smab-6 | 150 mg/ml secukinumab, 20 mM histidine, 200 mM mannitol |
| | dihydrate, 10 mM cysteine, 10 mM arginine 0.02% |
| | polysorbate 80, pH 5.8 |
| Smab-7 | 150 mg/ml secukinumab, 20 mM histidine, 200 mM trehalose |
| | dihydrate, 10 mM arginine 0.02% polysorbate 80, pH 5.8 |
| Smab-8 | 150 mg/ml secukinumab, 20 mM histidine, 200 mM mannitol, |
| | 10 mM arginine 0.02% polysorbate 80, pH 5.8 |
| Smab-9 | 150 mg/ml secukinumab, 20 mM histidine-acetate, 200 mM |
| | trehalose dihydrate, 10 mM di-sodium EDTA, 10 mM arginine |
| | 0.02% polysorbate 80, pH 5.8 |
| Smab-10 | 150 mg/ml secukinumab, 20 mM histidine-acetate, 200 mM |
| | mannitol dihydrate, 10 mM cysteine, 10 mM arginine 0.02% |
| | polysorbate 80, pH 5.8 |
| Smab-11 | 150 mg/ml secukinumab, 20 mM histidine-acetate, 200 mM |
| | trehalose dihydrate, 10 mM arginine 0.02% polysorbate 80, pH |
| | 5.8 |
| Smab-12 | 150 mg/ml secukinumab, 20 mM histidine-acetate, 200 mM |
| | mannitol, 10 mM arginine 0.02% polysorbate 80, pH 5.8 |
| Smab-13 | 150 mg/ml secukinumab, 20 mM histidine-citrate, 200 mM |
| | trehalose dihydrate, 10 mM di-sodium EDTA, 10 mM arginine |
| | 0.02% polysorbate 80, pH 5.8 |
| Smab-14 | 150 mg/ml secukinumab, 20 mM histidine-citrate, 200 mM |
| | mannitol dihydrate, 10 mM cysteine, 10 mM arginine 0.02% |
| | polysorbate 80, pH 5.8 |
| Smab-15 | 150 mg/ml secukinumab, 20 mM histidine-citrate, 200 mM |
| | trehalose dihydrate, 10 mM arginine 0.02% polysorbate 80, pH |
| | 5.8 |
| Smab-16 | 150 mg/ml secukinumab, 20 mM histidine-citrate, 200 mM |
| | mannitol, 10 mM arginine 0.02% polysorbate 80, pH 5.8 |

TABLE 4
SEC data of high concentration secukinumab
formulations prepared as per example 2

| Sam- | SEC data at 40° C. | | | | | | | | |
|---|---|---|---|---|---|---|---|---|---|
| ple | % of monomer | | | % of HMW | | | % LMW | | |
| Name | 0 W | 1 W | 2 W | 0 W | 1 W | 2 W | 0 W | 1 W | 2 W |
| Smab- | 98.4 | 94.6 | 93.1 | 1.6 | 3.4 | 4.2 | 0.05 | 1.95 | 2.7 |
| Control | | | | | | | | | |
| Smab-5 | 98.4 | 94.5 | 91.9 | 1.5 | 3.6 | 5.2 | 0.04 | 1.92 | 2.9 |
| Smab-6 | 98.6 | 91.9 | 88.0 | 1.3 | 3.7 | 6.6 | 0.4 | 4.41 | 5.4 |
| Smab-7 | 98.4 | 94.5 | 92.4 | 1.6 | 3.6 | 4.8 | 0.04 | 1.91 | 2.9 |
| Smab-8 | 98.3 | 94.7 | 92.0 | 1.6 | 3.4 | 5.2 | 0.05 | 1.88 | 2.8 |
| Smab-9 | 98.3 | 94.4 | 92.4 | 1.6 | 3.7 | 4.7 | 0.04 | 1.97 | 2.9 |
| Smab- | 98.5 | 91.1 | 87.3 | 1.4 | 4.9 | 7.3 | 0.1 | 3.97 | 5.3 |
| 10 | | | | | | | | | |
| Smab- | 98.3 | 94.3 | 92.2 | 1.7 | 3.7 | 5.0 | 0.05 | 1.99 | 2.8 |
| 11 | | | | | | | | | |
| Smab- | 98.2 | 94.1 | 92.3 | 1.7 | 3.7 | 4.7 | 0.05 | 2.13 | 3.0 |
| 12 | | | | | | | | | |
| Smab- | 98.5 | 94.7 | 92.4 | 1.5 | 3.4 | 4.8 | 0.04 | 1.87 | 2.8 |
| 13 | | | | | | | | | |
| Smab- | 98.8 | 94.0 | 91.2 | 1.2 | 2.6 | 4.3 | 0.06 | 3.35 | 4.6 |
| 14 | | | | | | | | | |
| Smab- | 98.4 | 94.8 | 93.5 | 1.5 | 3.4 | 4.0 | 0.04 | 1.79 | 2.5 |
| 15 | | | | | | | | | |
| Smab- | 98.4 | 94.8 | 93.0 | 1.6 | 3.4 | 4.4 | 0.04 | 1.79 | 2.6 |
| 16 | | | | | | | | | |

TABLE 5
IEX data of high concentration secukinumab
formulations prepared as per example 2

| | IEX data at 40° C. | | | | | | |
|---|---|---|---|---|---|---|---|
| Sam- | | | | % of main peak | | | |
| ple | % of acidic variants | | | | | | Slope/ |
| Name | 0 W | 1 W | 2 W | 0 W | 1 W | 2 W | week |
| Smab- | 30.3 | 36.6 | 42.4 | 64.2 | 50.7 | 45.5 | −6.8 |
| Control | | | | | | | |
| Smab-5 | 30.3 | 38.7 | 43.3 | 66.6 | 50.0 | 43.6 | −7.5 |
| Smab-6 | 39.6 | 56.2 | 56.0 | 55.0 | 34.4 | 16.1 | −19.5 |
| Smab-7 | 30.2 | 38.5 | 43.4 | 64.2 | 49.9 | 44.1 | −7.1 |
| Smab-8 | 30.6 | 35.1 | 41.9 | 64.0 | 52.7 | 45.5 | −6.6 |
| Smab-9 | 30.5 | 39.2 | 44.7 | 64.3 | 52.1 | 42.9 | −7.8 |
| Smab- | 40.6 | 63.9 | 58.6 | 53.7 | 34.4 | 14.1 | −18.6 |
| 10 | | | | | | | |
| Smab- | 30.6 | 39.7 | 44.5 | 63.8 | 50.8 | 43.6 | −7.1 |
| 11 | | | | | | | |
| Smab- | 30.7 | 36.4 | 43.8 | 63.9 | 49.1 | 44.0 | −7.0 |
| 12 | | | | | | | |
| Smab- | 30.7 | 40.3 | 44.7 | 66.8 | 50.4 | 42.6 | −8.2 |
| 13 | | | | | | | |
| Smab- | 30.7 | 40.3 | 44.7 | 59.5 | 41.5 | 28.3 | −15.6 |
| 14 | | | | | | | |
| Smab- | 30.8 | 38.1 | 43.8 | 63.7 | 49.9 | 44.5 | −7.2 |
| 15 | | | | | | | |
| Smab- | 30.5 | 37.8 | 44.1 | 64.0 | 51.3 | 43.9 | −7.1 |
| 16 | | | | | | | |

1. An aqueous high concentration pharmaceutical formulation of an anti-interleukin-17 antibody comprising about 150 mg/ml of anti-interleukin-17 antibody and a buffer comprising a combination of weak organic acid and organic base, having a pH of about 5.5 to about 6.5.

2. The formulation as claimed in claim 1, is devoid of anti-oxidant.

3. The formulation according to claim 1, further comprising one or more of the following excipients,
i) sugar,
ii) amino acid,
iii) surfactant.

4. The formulation as claimed in claim 3, wherein the sugar is trehalose or sucrose or mannitol.

5. The formulation as claimed in claim 3, wherein the amino acid is arginine.

6. A method of inhibiting charge variants formation and/or aggregation of an anti-interleukin-17 antibody in the formulation composition of the antibody, the method comprising, formulating the antibody in a weak organic acid and organic base buffer composition, having a pH of about 5.5 to 6.5, and comprising, sugar and arginine, and wherein the method does not require an anti-oxidant.

7. (canceled)

8. A method of controlling oxidation of an anti-interleukin-17 antibody in the formulation composition of the antibody, the method comprising, formulating the antibody in a weak organic acid and organic base buffer composition, having a pH of about 5.5 to 6.5, and comprising, sugar and arginine, and wherein the method does not require an anti-oxidant.

9. The formulation as claimed in claim 1, wherein the organic base is histidine.

10. The formulation as claimed in claim 1, wherein the weak organic acid is acetate or citrate.

11. The formulation as claimed in claim 2 wherein the anti-oxidant is methionine.

12. The formulation as claimed in claim 6 wherein the anti-oxidant is methionine.

13. The formulation as claimed in claim 8 wherein the anti-oxidant is methionine.
